(19) United States
(12) Patent Application Publication
(10) Pub. No.: US 2025/0120358 A1
(43) Pub. Date: Apr. 17, 2025

(54) METHOD FOR IMPROVING DENSITY-TOLERANT YIELD OF MAIZE AND USE THEREOF

(71) Applicants: BIOTECHNOLOGY RESEARCH INSTITUTE OF CHINESE ACADEMY OF AGRICULTURAL SCIENCES, Beijing (CN); SOUTH CHINA AGRICULTURAL UNIVERSITY, Guangzhou (CN)

(72) Inventors: Baobao WANG, Beijing (CN); Haiyang WANG, Guangzhou (CN); Changhe SUN, Beijing (CN); Di YANG, Beijing (CN); Zhanchao XIA, Beijing (CN); Yuelei ZHAO, Beijing (CN); Xinan ZHOU, Beijing (CN); Xinle ZHU, Beijing (CN); Zhigang ZHENG, Guangzhou (CN); Yurong XIE, Beijing (CN); Miaoyun XU, Beijing (CM)

(73) Assignees: BIOTECHNOLOGY RESEARCH INSTITUTE OF CHINESE ACADEMY OF AGRICULTURAL SCIENCES, Beijing (CN); SOUTH CHINA AGRICULTURAL UNIVERSITY, Guangzhou (CN)

(21) Appl. No.: 18/901,612

(22) Filed: Sep. 30, 2024

Related U.S. Application Data
(63) Continuation of application No. PCT/CN2024/074729, filed on Jan. 30, 2024.

(30) Foreign Application Priority Data
Jun. 26, 2023 (CN) 202310754927.3

(51) Int. Cl. A01H 1/00

(52) U.S. Cl. CPC A01H 1/12

(57) ABSTRACT

The present application discloses a method for improving density-tolerant yield of maize and use thereof, belongs to the field of plant biotechnology breeding. Compared with the Ig1 mutant strain, the LG1/Ig1 heterozygous genotype strain has excellent agronomic characters such as a widened leaf, a larger spike, a reduced empty stalk ratio and/or an increased yield by close planting; compared with the LG1 wild-type strain, the LG1/Ig1 heterozygous genotype material reduces a leaf angle and tassel branch number of maize, and has the effect of increasing the yield by close planting. The method has a wide application prospect in the field of maize high-yield breeding.

### RELATED APPLICATIONS

The present patent document is a continuation of PCT Application Serial No. PCT/CN2024/074729, filed Jan. 30, 2024, designating the United States and published in English, which is hereby incorporated by reference. The present patent document claims the benefit of priority to patent application No. 202310754927.3, filed Jun. 26, 2023, the entire contents of each of which are incorporated herein by reference.

### TECHNICAL FIELD

The present application belongs to the field of plant biotechnology breeding, and in particular relates to a method for improving density-tolerant yield of maize by using an LG1/Ig1 heterozygous genotype and use thereof.

### BACKGROUND

Studies show that enhancing the density-tolerance and planting density of varieties is the key to enhance the yield of maize per unit area. In the past 80 years, the planting density of maize in the United States has enhanced from about 30000 plants/hectare in 1930s to about 70000 plants/hectare now (62000-104000 plants/hectare); at the same time, the yield of maize per unit area has enhanced from 1287 kilograms/hectare in 1930s to 9595 kilograms/hectare in 2010 (USDA-NASS, 2012); however, in this process, the increase in yield of maize per plant and heterosis is not obvious, and the increase in yield per unit area is more due to the continuous increase in density-tolerance and planting density of varieties. Studies on maize varieties in different years in China also show that compared with early varieties, modern varieties are developing in a more density-tolerant direction in photosynthetic efficiency, lodging resistance, empty stalk ratio, yield, etc. Therefore, enhancing the density-tolerance and planting density of varieties is an important goal and trend in modern maize breeding and production.

Studies find that LG1 gene is the key factor to regulate the change of leaf angle of gramineous crops such as maize, rice and wheat. Studies have shown that homozygous Ig1 can reduce both the leaf angle and the tassel branch number, which plant type is beneficial for a density-tolerant condition. However, the inventors find that it lacks the auricle and ligule, which is easy to cause pests and pathogens to invade from sheaths and increase the risk of pathogen and pest damages, and previous experiments have showed that the homozygous Ig1 mutant did not have the effect of increasing yield under high density conditions (5000 plants/mu and 6000 plants/mu, reference: Lambert RJ, Johnson RR: Leaf Angle, Tassel Morphology, and the Performance of Maize Hybrids. Crop Science. 1978, 18 (3): 499-502). Therefore, the breeding strategies and methods which can reduce a leaf angle and a tassel branch number, but can normally produce an auricle and a ligule, and at the same time, will not make a spike smaller significantly, and not increase an empty stalk ratio during production will have broad use prospects in the field of density-tolerant breeding of maize.

### BRIEF SUMMARY

All references mentioned herein are incorporated herein by reference. Unless otherwise indicated, all technical and scientific terms used herein have the same meaning as commonly understood by those of ordinary skill in the art to which the present application belongs. Unless otherwise indicated, the techniques used or mentioned herein are standard techniques known to those of ordinary skill in the art. The materials, methods and examples are for illustration only, not for limitation.

The examples of the present application provide an improved density-tolerant breeding method, including obtaining an LG1/Ig1 heterozygous genotype strain by hybridizing an Ig1 mutant strain with an LG1 wild-type strain, wherein the heterozygous genotype strain has excellent density-tolerant characters.

Optionally, the excellent density-tolerant characters include: a reduced maize leaf angle, a reduced tassel branch number, a reduced tassel branch angle, a widened leaf, a larger spike, a reduced empty stalk ratio and/or an increased yield by close planting. Specifically, compared with the LG1 wild-type strain, the LG1/Ig1 heterozygous genotype material has a reduced maize leaf angle, a reduced tassel branch number, and an increased yield by close planting; compared with the Ig1 mutant, the LG1/Ig1 heterozygous genotype material has a widened leaf, a larger spike, a reduced empty stalk ratio, an increased tassel branch angle, an increased yield by close planting and/or an enhanced ability to resist pathogen and pest damages. Those skilled in the art have known that widening of a plant leaf can make the leaves intercept sunlight more effectively, which is beneficial for photosynthesis and further beneficial for the enhancement of the yield.

Optionally, the polynucleotide sequence of the wild-type LG1 gene is selected from one of the sequences in following groups:
(a) a polynucleotide sequence as shown in SEQ ID No: 1, 2 or 3;
(b) a polynucleotide sequence encoding an amino acid sequence as shown in SEQ ID No: 4;
(c) a polynucleotide sequence which can hybridize with the polynucleotide sequence in (a) or (b) under stringent hybridization conditions, wherein the homozygous mutation of the polynucleotide sequence has functions of completely deleting a ligule and an auricle, reducing a leaf angle, making a leaf erect, narrowing a leaf, reducing a tassel branch number, reducing a tassel branch angle, raising an empty stalk ratio and/or making a spike smaller in a plant;
(d) a polynucleotide sequence which has at least 90%, 95%, 98% or more sequence identity to the polynucleotide sequence as shown in any one of (a) to (c), wherein the homozygous mutation of the polynucleotide sequence has functions of completely deleting a ligule and an auricle, reducing a leaf angle, making a leaf erect, narrowing a leaf, reducing a tassel branch number, reducing a tassel branch angle, raising an empty stalk ratio and/or making a spike smaller in a plant; or
(e) a polynucleotide sequence which is fully complementary to the sequence of any one of (a) to (d).

Optionally, the LG1 gene provided by the examples of the present application further includes a homologous gene which has at least 80%, 85%, 90%, 95%, 98% or 99% sequence identity to the polynucleotide sequence disclosed by the examples of the present application, or a homologous gene which has at least 90%, 95% or 98% sequence identity to the amino acid sequence of LG1 disclosed by the example of the present application. And after endogenous homozygous mutation, the homologous gene has functions of completely deleting a ligule and an auricle, reducing a leaf angle, making a leaf erect, narrowing a leaf, reducing a tassel branch number, reducing a tassel branch angle, raising an empty stalk ratio and/or making a spike smaller in a plant. The homologous gene can be isolated from any plant.

Optionally, the method provided by the examples of the present application can be used in any plant containing an LG1 homologous gene. Preferably, the plant includes monocotyledon such as maize, millet, wheat, barley, rye, rice and sorghum.

The percentage of sequence identity described in the present application can be obtained by commonly known bioinformatics algorithms, including the Myers and Miller algorithm, the Needleman-Wunsch global alignment method, the Smith-Waterman local alignment method, the Pearson and Lipman similarity search method, the Karlin and Altschul algorithm, which are commonly known to those skilled in the art.

Those skilled in the art should know that there is Single Nucleotide Polymorphism (SNP) in the same gene among different varieties of the same plant, i.e., the nucleotide sequence of the same gene often has individual base differences, but there are so many varieties in the same crop that it is impossible for the inventors to list them one by one. The examples of the present application only provide the sequences of representative varieties in maize crops. Therefore, those skilled in the art should know that the nucleotide sequences from different varieties with SNP with the LG1 gene and its nucleotide sequence disclosed in the present application, and the method and use which utilize the homozygous mutant strain obtained by mutation of the nucleotide sequences to hybridize with the wild-type strain to obtain characters such as a widened leaf, a larger spike, a reduced empty stalk ratio and/or an increased yield by close planting, are also within the protection scope of the present application.

Optionally, the Ig1 mutant strain is obtained by mutation including substitution, deletion and/or addition of one or more nucleotides on the nucleotide sequence of the gene.

Optionally, the mutation is obtained by technologies such as physical mutagenesis, chemical mutagenesis, ZFN, TALEN and/or CRISPR/Cas gene editing.

Optionally, when using CRISPR/Cas for maize plant gene editing, the target site sequence is shown in SEQ ID NO:5 or SEQ ID NO:6.

Optionally, the Ig1 mutant has a genomic mutant nucleotide sequence such as one of the sequences in following groups:
(a) a genomic mutant nucleotide sequence in which the whole nucleotide sequence of SEQ ID No.1 is deleted;
(b) a genomic mutant nucleotide sequence in which the nucleotide sequence at 743-764 bp of SEQ ID No. 1 or SEQ ID No.2 or the nucleotide sequence at 229-250 bp of SEQ ID No.3 is replaced by 5′-TTCCGCCGCCGTCC-3′;
(c) a genomic mutant nucleotide sequence in which a sequence deletion occurs at the nucleotide sequence at 766-835 bp of SEQ ID No. 1 or SEQ ID No.2, or at 252-321 bp of SEQ ID No.3; and
(d) a genomic mutant nucleotide sequence in which a single nucleotide deletion occurs at the nucleotide sequence at 836 bp of SEQ ID No. 1 or SEQ ID No.2, or at 322 bp of SEQ ID No.3.

Optionally, the close planting means the planting density of maize which is more than or equal to 4000 plants/mu, preferably more than or equal to 5000 plants/mu.

Optionally, the examples of the present application further provide use of any of the methods mentioned above in maize breeding, preferably including but not limited to use in reducing a leaf angle, widening a leaf, reducing a tassel branch number, reducing a tassel branch angle, reducing an empty stalk ratio and/or increasing a yield by close planting in a plant.

Specifically, the increased yield by close planting therein means increasing a maize yield under high density planting conditions, and the high density means more than or equal to 4000 plants/mu, preferably, more than or equal to 5000 plants/mu.

The examples of the present application further provide a plant cell, a tissue, an organ or a commercial product not used as a propagation material, which contains a heterozygous LG1/Ig1 genotype. The heterozygous LG1/Ig1 genotype means the plant cell, tissue, organ or commercial product containing both the wild-type LG1 gene and the mutant Ig1.

The examples of the present application further provide a method for regulating and controlling agronomic characters of a maize plant, including obtaining a homozygous mutant through mutating an endogenous gene of the plant, wherein the homozygous mutant has phenotypes of a completely deleted ligule and auricle, a reduced leaf angle, an erect leaf, a narrowed leaf, a reduced tassel branch number, a reduced tassel branch angle, a raised empty stalk ratio and/or a smaller spike, and the polynucleotide sequence of the endogenous gene is selected from one of the sequences in following groups:
(a) a polynucleotide sequence as shown in SEQ ID No: 1, 2 or 3;
(b) a polynucleotide sequence encoding an amino acid sequence as shown in SEQ ID No: 4;
(c) a polynucleotide sequence which can hybridize with the polynucleotide sequence in (a) or (b) under stringent hybridization conditions, wherein the homozygous mutation of the polynucleotide sequence has functions of completely deleting a ligule and an auricle, reducing a leaf angle, making a leaf erect, narrowing a leaf, reducing a tassel branch number, reducing a tassel branch angle, raising an empty stalk ratio and/or making a spike smaller in a plant;
(d) a polynucleotide sequence which has at least 90%, 95%, 98% or more sequence identity to the polynucleotide sequence as shown in any one of (a) to (c), wherein the homozygous mutation of the polynucleotide sequence has functions of completely deleting a ligule and an auricle, reducing a leaf angle, making a leaf erect, narrowing a leaf, reducing a tassel branch number, reducing a tassel branch angle, raising an empty stalk ratio and/or making a spike smaller in a plant; or
(e) a polynucleotide sequence which is fully complementary to the sequence of any one of (a) to (d).

Optionally, transferring, or introducing, or transforming a nucleotide sequence, a vector, a construct or an expression cassette into a plant mentioned in the examples of the present application all means transferring a target nucleotide sequence, a construct, a vector or an expression cassette into a receptor cell or a receptor plant by a conventional transgenic method or a method of hybridization with the target transgenic plant. Any transgenic method known to those skilled in the art can be used to transform recombinant expression vectors into plant cells to produce transgenic plants or mutants of the examples of the present application. Transformation methods can include direct or indirect transformation methods. Specifically, the transformation methods include but not limited to polyethylene glycol-induced DNA uptake, liposome-mediated transformation, gene gun introduction, electroporation, microinjection, and the agrobacterium-mediated plant transformation method.

Compared with the prior art, the present application has the following beneficial effects:

(1) The examples of the present application provide a method for improving density-tolerant yield of maize and use thereof, including: hybridizing an Ig1 mutant strain with an LG1 wild-type strain to obtain an LG1/Ig1 heterozygous genotype strain, wherein compared with the Ig1 mutant strain, the LG1/Ig1 heterozygous genotype strain has excellent agronomic characters such as a widened leaf, a larger spike, a reduced empty stalk ratio and/or an increased yield by close planting; compared with the LG1 wild-type strain, the LG1/Ig1 heterozygous genotype material reduces a leaf angle and tassel branch number of maize, and has the effect of increasing the yield by close planting; in the density-tolerant high-yield breeding, the technical problems that an Ig1 mutant has density-tolerant characters of a smaller leaf angle and an erect leaf, but has a reduced yield, a reduced ability to resist pathogen and pest damages, and an increased empty stalk ratio are solved.

(2) The method provided by the examples of the present application provides the possibility for cultivating new crop varieties with a density-tolerance and a high yield, especially new maize varieties, and is of great significance to global food security and sustainable agricultural development.

### Definitions of Terms Involved in the Present Application

Unless otherwise defined, all technical and scientific terms used herein have the same meaning as commonly understood by those of ordinary skill in the art to which the present application belongs. Although any methods, devices and materials similar or equivalent to those described herein can be used in the practice or testing of the present application, preferred methods, devices and materials are now described.

In the context of the present application, the term “polynucleotide” or “nucleotide” means deoxyribonucleotide, deoxyribonucleoside, ribonucleoside or ribonucleotide and polymers thereof in single or double stranded form. Unless specifically limited, the term covers nucleic acids containing known analogs of natural nucleotides, which have binding properties similar to those of reference nucleic acids and are metabolized in a manner similar to naturally occurring nucleotides.

In the present application, the term “homologous gene” means two or more gene sequences with a sequence identity of 80%, including orthologous genes (also called vertical homologous genes, orthologous genes or directed evolutionary homologous genes), transverse homologous genes (also called paralogous genes, paraphyletic homologous genes or parallel evolutionary homologous genes) and/or heterologous homologous genes.

The term sequence “identity” means the identity between two sequences, and it is a quantitative concept, which is used to compare the identity between different sequences, so as to find and analyze the correlation between two sequences. A sequence identity can be used to compare gene sequences, protein sequences, DNA sequences, etc. The “stringent hybridization condition” mentioned in the present application means conditions of low ionic strength and high temperature known in that art. Generally, under stringent conditions, the detectable degree of hybridization between a probe and its target sequence is higher than that of hybridization with other sequences (for example, it exceeds the background by at least 2 times). Stringent hybridization conditions are sequence-dependent, which will be different under different environmental conditions. Longer sequences hybridize specifically at higher temperatures. By controlling the stringency of hybridization or washing conditions, the target sequence which is 100% complementary to the probe can be identified. For detailed instructions on nucleic acid hybridization, please refer to relevant literature (Tijssen, Techniques in Biochemistry and Molecular Biology-Hybridization with Nucleic Probes, “Overview of principles of hybridization and the strategy of nucleic acid assays. 1993). More specifically, the stringent conditions are usually selected to be about 5-10° C. lower than the hot melting point (Tm) of the specific sequence at a specified ionic strength pH. Tm is the temperature at which 50% of the probes complementary to the target hybridize to the target sequence in the equilibrium state (at a specified ionic strength, pH and nucleic acid concentration) (50% of the probes are occupied in the equilibrium state at Tm because the target sequence exists in excess). Stringent conditions may be those in which the salt concentration is lower than about 1.0 M sodium ion concentration at pH 7.0 to 8.3, usually about 0.01 to 1.0 M sodium ion concentration (or other salts), and the temperature is at least about 30° C. for short probes (including but not limited to 10 to 50 nucleotides) and at least about 60° C. for long probes (including but not limited to more than 50 nucleotides). Stringent conditions can also be achieved by adding destabilizers such as formamide. For selective or specific hybridization, the positive signal can be at least 2 times that of background hybridization, and optionally 10 times that of background hybridization. Exemplary stringent hybridization conditions can be as follows: 50% formamide, 5×SSC and 1% SDS, cultured at 42° C.; or 5×SSC, 1% SDS, cultured at 65° C., washed in 0.2×SSC and washed in 0.1% SDS at 65° C. The washing may be carried out for 5, 15, 30, 60, 120 minutes or longer.

The term “recombinant expression vector”: one or more DNA vectors used for plant transformation; these vectors are often referred to as binary vectors in the art. Binary vectors together with vectors with auxiliary plasmids are mostly and commonly used for agrobacterium-mediated transformation. Binary vectors usually include: cis-acting sequences needed for T-DNA transfer, selectable markers engineered to be expressed in plant cells, heterologous DNA sequences to be transcribed, etc.

The term “leaf angle” means the angle formed by the midrib of a leaf and the upper stalk contacted by the pulvinus of the leaf.

The term “hybridization” is a broad hybridization, which means the process of gamete combining between individuals of different populations or genotypes to produce hybrids. According to the difference of parental genetic relationships, hybridization includes close hybridization and distant hybridization.

In the present application, an Ig1 mutant, an Ig1 homozygous mutant or an Ig1/Ig1 mutant all mean an Ig1 homozygous mutant.

### BRIEF DESCRIPTION OF THE DRAWINGS

FIG. 1 is the comparative analysis of a wild-type and an Ig1 mutant. Wherein,

FIG. 1A is the sequences of a B73 inbred line and an obtained Ig1 mutant under the background of B73, in which LG1-WT is the B73 wild-type, and LG1-MT is the Ig1 mutant under the background of B73;

FIG. 1B is the comparison of the whole plant types of the B73 inbred line and the Ig1 mutant under the background of B73;

FIG. 1C is the comparison of the leaf angles of the B73 inbred line and the Ig1 mutant under the background of B73; and

FIG. 1D is the comparison of the auricles and ligules of the B73 inbred line and the Ig1 mutant under the background of B73.

FIG. 2 is the comparative analysis of the characters of an LG1 wild-type material, a heterozygous material and a mutant homozygous material. Wherein,

FIG. 2A is the comparative photograph of the whole plant types of an LG1/LG1 wild-type material, an LG1/Ig1 heterozygous genotype material and an Ig1/Ig1 homozygous mutant material under the background of B73;

FIG. 2B and FIG. 2E are the comparative photographs and the statistics of the leaf angles of the LG1/LG1 wild-type material, the LG1/Ig1 heterozygous genotype material and the Ig1/Ig1 homozygous mutant material under the background of B73, respectively;

FIG. 2C and FIG. 2F are the comparative photographs of the tassels and the statistics of the tassel branch numbers of the LG1/LG1 wild-type material, the LG1/Ig1 heterozygous genotype material and the Ig1/Ig1 homozygous mutant material under the background of B73;

FIG. 2D and FIG. 2G are the comparative photographs of the spikes and the statistics of the spike weights of the LG1/LG1 wild-type material, the LG1/Ig1 heterozygous genotype material and the Ig1/Ig1 homozygous mutant material under the background of B73.

FIG. 3 is the mutation of the LG1 gene under the background of the obtained PH4CV, PH6WC and Jing724 maize inbred lines.

FIG. 4 is the comparative photographs of the leaf angles of the LG1/LG1 wild-type material, the LG1/Ig1 heterozygous genotype material and the Ig1/Ig1 homozygous mutant material under the background of the PH6WC, PH4CV and B73 inbred lines under different density conditions and the statistics thereof. LD: 3000 plants/mu, MD: 6000 plants/mu, and HD: 9000 plants/mu.

FIG. 5 is the comparative photographs of the tassels and the statistics of the tassel branch numbers of the LG1/LG1 wild-type material, the LG1/Ig1 heterozygous genotype material and the Ig1/Ig1 homozygous mutant material under the background of the PH4CV and PH6WC inbred lines under different density conditions. LD: 3000 plants/mu, MD: 6000 plants/mu, and HD: 9000 plants/mu.

FIG. 6 is the photographs of the leaf angles of the LG1/LG1 wild-type material, the LG1/Ig1 heterozygous genotype material and the Ig1/Ig1 homozygous mutant material under the background of the hybrids B73/PH6WC, B73/PH4CV and PH6WC/PH4CV under different density conditions and the statistics thereof. LD: 3000 plants/mu, MD: 5000 plants/mu, and HD: 7000 plants/mu.

FIG. 7 is the comparative photographs of the tassels and the statistics of the tassel branch numbers of the LG1/LG1 wild-type material, the LG1/Ig1 heterozygous genotype material and the Ig1/Ig1 homozygous mutant material under the background of the hybrids B73/PH6WC and B73/PH4CV under different density conditions. LD: 3000 plants/mu, MD: 5000 plants/mu, and HD: 7000 plants/mu.

FIG. 8 is the statistical analysis of the empty stalk ratios of different strains under different density conditions. Wherein, FIG. 8A, FIG. 8B and FIG. 8C are the statistics of the empty stalk ratios of the LG1/LG1 wild-type material, the LG1/Ig1 heterozygous genotype material and the Ig1/Ig1 homozygous mutant material under the background of the B73, PH4CV and PH6WC inbred lines under different density conditions, respectively; FIG. 8D, FIG. 8E and FIG. 8F are the statistics of the empty stalk ratios of the LG1/LG1 wild-type material, the LG1/Ig1 heterozygous genotype material and the Ig1/Ig1 homozygous mutant material under the background of the hybrids B73/PH4CV, B73/PH6WC and PH6WC/PH4CV under different density conditions, respectively. LD: 3000 plants/mu, MD: 6000 plants/mu, and HD: 9000 plants/mu.

FIG. 9 is the statistics of the plot yields of the LG1/LG1 wild-type material, the LG1/Ig1 heterozygous genotype material and the Ig1/Ig1 homozygous mutant material under the background of the hybrids B73/PH6WC, B73/PH4CV and PH6WC/PH4CV under different density conditions.

FIG. 10 is the statistical analysis of the leaf widths under the background of different inbred lines and hybrids. Wherein, FIG. 10A and FIG. 10B are the statistics of the leaf widths of the LG1/LG1 wild-type material, the LG1/Ig1 heterozygous genotype material and the Ig1/Ig1 homozygous mutant material under the background of the PH4CV and PH6WC inbred lines under different density conditions, respectively; FIG. 10C and FIG. 10D are the statistics of the leaf widths of the LG1/LG1 wild-type material, the LG1/Ig1 heterozygous genotype material and the Ig1/Ig1 homozygous mutant material under the background of the hybrids B73/PH4CV and PH6WC/PH4CV under different density conditions, respectively. LD: 3000 plants/mu, MD: 6000 plants/mu, and HD: 9000 plants/mu.

FIG. 11 is the statistics of different phenotypes of the LG1/LG1 wild-type material and the LG1/Ig1 heterozygous genotype material under the background of the hybrids Jing724/Jing92 and Jing724-Ig1/Jing92 under the planting conditions of 3000 plants in the field. Wherein,

FIG. 11A is the plant types of the LG1/LG1 wild-type material and the LG1/Ig1 heterozygous genotype material under the background of the hybrids Jing724/Jing92 and Jing724-Ig1/Jing92 under the condition of 3000 plants/mu in the field;

FIG. 11B is the tassel photographs of the LG1/LG1 wild-type material and the LG1/Ig1 heterozygous genotype material under the background of the hybrids Jing724/Jing92 and Jing724-Ig1/Jing92 under the condition of 3000 plants/mu in the field (compared with the LG1/LG1 wild-type material, the LG1/Ig1 heterozygous genotype material has a reduced tassel branch number and a reduced tassel angle);

FIG. 11C is the statistics of the plot yields of the LG1/LG1 wild-type material and the LG1/Ig1 heterozygous genotype material under the background of the hybrids Jing724/Jing92 and Jing724-Ig1/Jing92 under different density conditions in the field.

### DETAILED DESCRIPTION

The present application will be further described with specific examples, and the advantages and characteristics of the present application will become clearer with the description. However, these examples are only exemplary and do not limit the scope of the present application in any way. It should be understood by those skilled in the art that the details and forms of the present application can be modified or substituted without departing from the spirit and scope of the present application, but these modifications and substitutions are all within the protection scope of the present application.

The inbred lines used in the following examples can obtain relevant information from “Chinese Crop Germplasm Resources Information System” and apply for the corresponding seeds.

### Example 1. Rapid Creation of Ig1 Gene Mutant Under Background of B73 Inbred Line by “Haploid Mediated Gene Editing Technology (IMGE)”

In the early stage, the inventor skillfully combined haploid induction with gene editing technology, and developed “haploid mediated gene editing technology (IMGE)” (reference: Baobao Wang et al., Development of a Haploid-Inducer Mediated Genome Editing System for Accelerating Maize Breeding, Molecular Plant 12, 597-602, April 2019). The technical route of the method is as follows: introducing a CRISPR/Cas9 vector into a haploid induction line by a backcross or genetic transformation method, then hybridizing the haploid induction line carrying the CRISPR/Cas9 vector as a paternal plant with a commercial inbred line to obtain haploids, and screening haploids with edited target sites according to the change of target characters and sequencing analysis, and finally obtaining homozygous and gene-edited dihaploid materials by artificial or natural chromosome doubling, so as to create commercial germplasm dihaploid lines with improved characters and no transgenic ingredients within two generations (one year), which greatly accelerated the process of crop breeding. The IMGE technology breaks through the problem that traditional gene editing is limited by the genetic background and genetic transformation ability of the materials to be improved, and can achieve directional improvement of agronomic characters within two generations, which is rapid and efficient. At the same time, the improved materials do not carry exogenous transgenic ingredients, so they are easy for commercial development, especially friendly to breeders and convenient for operation: gene editing can be achieved just through pollination.

In order to rapidly create a Ig1 mutant under the background of B73 by using the IMGE technology, we constructed a gene editing vector targeting the LG1 gene (the target sites were SEQ ID No.5: 5′-GCGGAGACTAAGTGGCTGTAGGG-3′ and SEQ ID No.6:5 ‘-GGGGGAGCATCACCATCAACTGC-3’), and genetically transformed the vector into a ZC01 inbred line. The obtained transgenic event with a high mutation rate was hybridized with the maize haploid inducing line CAU5, and then backcrossed with CAU5 for three generations to obtain a maize haploid inducing line-CAU5LG1-IMGE with a LG1 gene editing vector in the BC3F1 generation. There were two key points in this process: one was to use the Bar gene on the gene vector as a screening marker to ensure that the gene editing vector targeting the LG1 gene remained in the backcross offsprings, and the other was to ensure that the haploid inducing alleles Zmmtl and Zmdmp from CAU5 remained in the backcross offsprings in a homozygous state through sequencing identification.

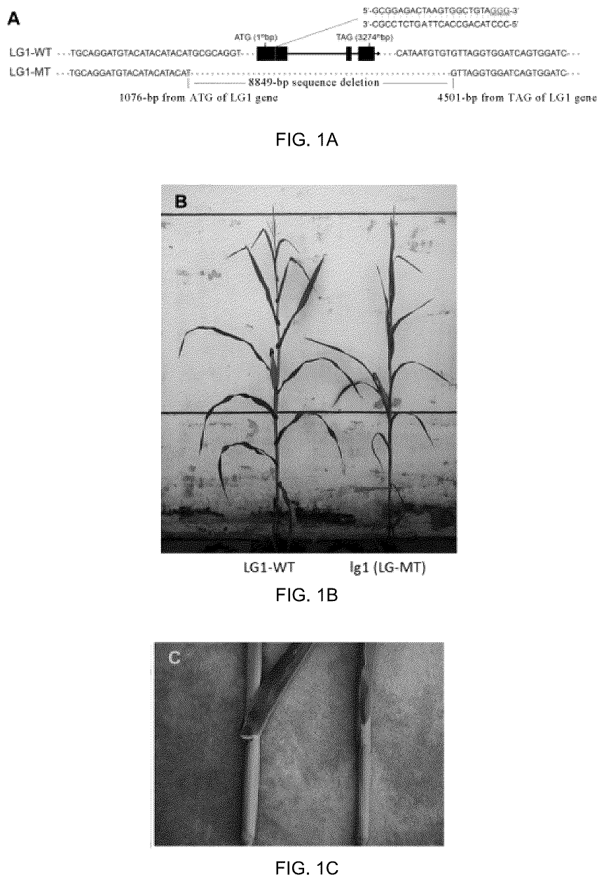

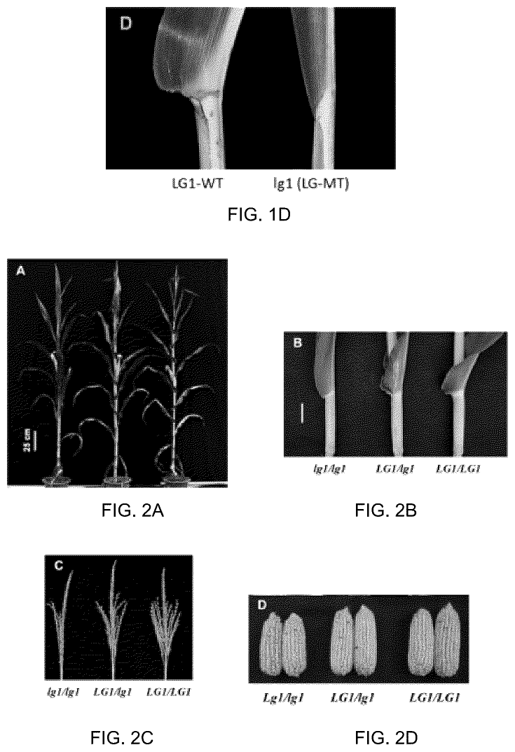

Then, CAU5LG1-IMGE was used to carry out the IMGE haploid induction on a B73 inbred line, to obtain a haploid containing the Ig1 mutation successfully, which was natural doubled to obtain a Ig1 mutant under the background of the B73 inbred line (FIG. 1), containing no exogenous transgenic ingredients (containing no CRISPR vector). Sequencing analysis shows that the 8.8 kb sequence comprising the whole coding region of the LG1 gene in the mutant is deleted (FIG. 1A), and the rest of the genetic background is almost the same as B73. Phenotype analysis shows that the ligule and auricle of the B73-Ig1 homozygous mutant are completely deleted, leading to a reduced leaf angle and an erect leaf (FIG. 1B-FIG. 1D).

### Example 2. Phenotype Analysis of LG1/LG1 Wild-Type Material, LG1/Ig1 Heterozygous Genotype Material and Ig1/Ig1 Homozygous Mutant Material Under Background of B73

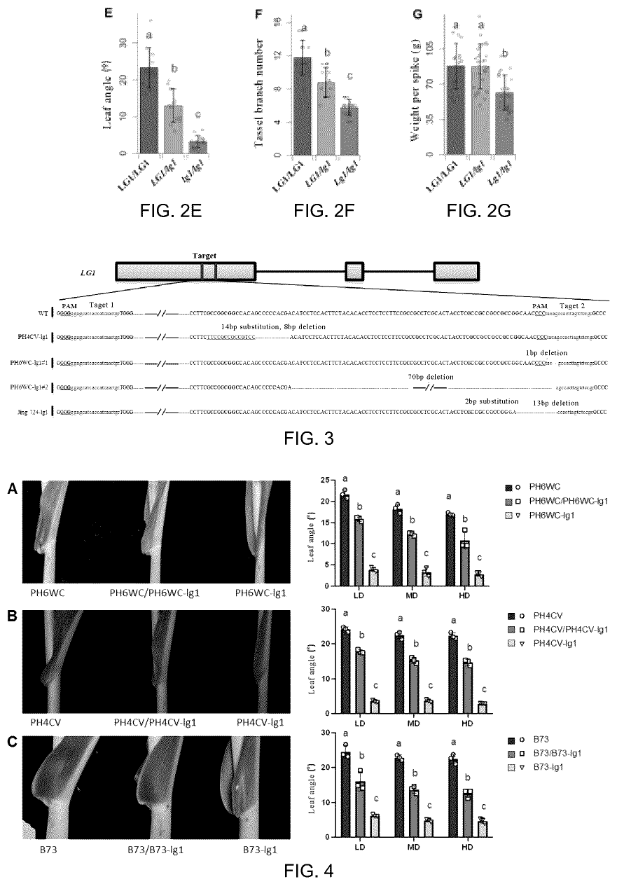

Comparing B73 with the Ig1 homozygous mutant under the background of B73, it is found that the homozygous Ig1/Ig1 mutant has not only a deleted auricle and ligule, and a reduced leaf angle, but also a narrowed leaf (FIGS. 1C and 1D), a reduced tassel branch number and a much smaller spike (FIG. 2). Generally, reducing both the leaf angle and the tassel branch number of a plant is beneficial for density-tolerance of maize in plant type. However, the Ig1 homozygous mutant lacks the auricle and ligule, which can easily cause pests and pathogens to invade from sheaths and increase the risk of pathogen and pest damages, and it has a smaller spike. These factors are extremely unfavorable to the high yield and stable yield of maize under close planting conditions, indicating that the Ig1/Ig1 homozygous mutant cannot really achieve the use of density-tolerant breeding.

Then, we created an LG1/Ig1 heterozygous genotype material under the background of B73. With the analysis of the LG1/LG1 wild-type material and the Ig1/Ig1 homozygous mutant material, it is shown (FIG. 2) that under the normal planting density of 4500 plants/mu, the LG1/Ig1 heterozygous genotype material has a leaf angle and a tassel branch number which are both between those of the Ig1/Ig1 homozygous mutant material and the LG1/LG1 wild-type material; while having a normal auricle and ligule, thus can effectively prevent pests and pathogens from invading sheaths. More interestingly, we find that under the condition of 4500 plants/mu, the LG1/Ig1 heterozygous genotype material has a spike size that is almost the same as that of the homozygous LG1/LG1 wild-type material, and does not show the effect of making the spike smaller and yield reduction. The result shows that the material has a compacter leaf angle, and a reduced tassel branch number, and does not have a smaller spike, which indicates that the LG1/Ig1 heterozygous genotype material has an unexpected use potential for density-tolerant breeding.

### Example 3. Test of Density-Tolerant Effect of LG1/LG1 Wild-Type Material, LG1/Ig1 Heterozygous Genotype Material and Ig1/Ig1 Homozygous Mutant Material Under Background of a Plurality of Inbred Lines

In order to test the use potential and universality of an LG1/Ig1 heterozygous genotype material in density-tolerant breeding, the maize haploid inducing line CAU5LG1-IMGE containing the LG1 gene editing vector created earlier was used to carry out the IMGE haploid induction on the key maize inbred lines PH6WC, PH4CV and Jing724, to obtain haploids containing the Ig1 mutation successfully, which were natural doubled to obtain Ig1 mutants under the background of the PH6WC, PH4CV and Jing724 inbred lines. Wherein, one Ig1 gene mutant under the background of PH4CV was obtained (FIG. 3), and named as PH4CV-Ig1, which had a mutation site sequence shown in FIG. 3, and had a genome mutant nucleotide sequence characterized in that the nucleotide sequence at 743-764 bp of SEQ ID No.1 or SEQ ID No.2 or the nucleotide sequence at 229-250 bp of SEQ ID No.3 was replaced by 5′-TTCCGCCGCCGTCC-3′ (SEQ ID NO:7); two Ig1 gene mutants under the background of PH6WC were obtained (FIG. 3), and named as PH6WC-Ig1 #1 and PH6WC-Ig1 #2, respectively, which had mutation site sequences shown in FIG. 3, wherein PH6WC-Ig1 #1 had a genome mutant nucleotide sequence characterized in that a single nucleotide deletion occurs at the nucleotide sequence at 836 bp of SEQ ID No. 1 or SEQ ID No.2, or at 322 bp of SEQ ID No.3; PH6WC-Ig1 #2 had a genome mutant nucleotide sequence characterized in that a sequence deletion occurs at the nucleotide sequence at 766-835 bp of SEQ ID No. 1 or SEQ ID No.2, or at 252-321 bp of SEQ ID No.3; one Ig1 gene mutant under the background of Jing724 was obtained (FIG. 3), and named as Jing724-Ig1, which had a mutation site sequence shown in FIG. 3, and had a genome mutant nucleotide sequence characterized in that the sequence at 822-837 bp of SEQ ID No. 1 or SEQ ID No.2 was replaced by the genome mutant nucleotide sequence of 5′-GGA-3′. Then, LG1/Ig1 heterozygous genotype materials under the background of PH6WC and PH4CV inbred lines were created by hybridization, wherein PH6WC was created with PH6WC-Ig1 #1.

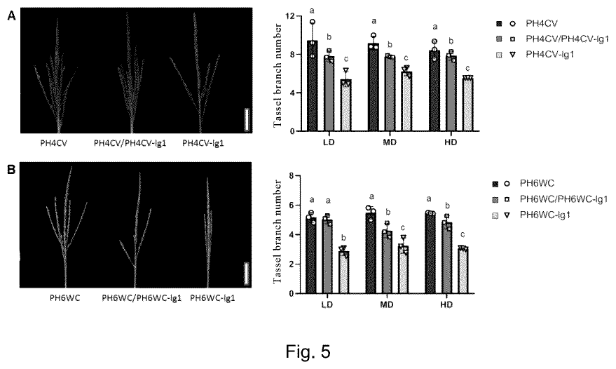

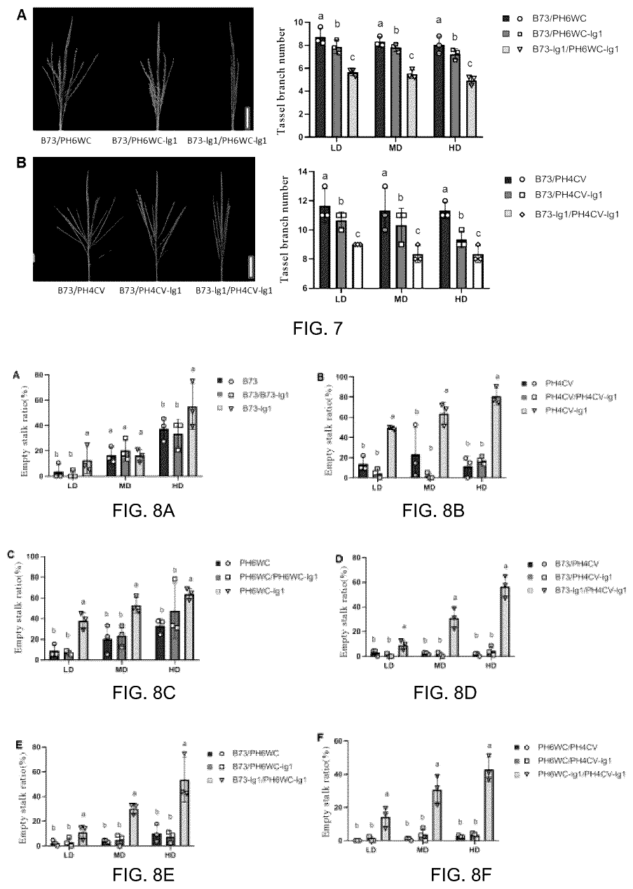

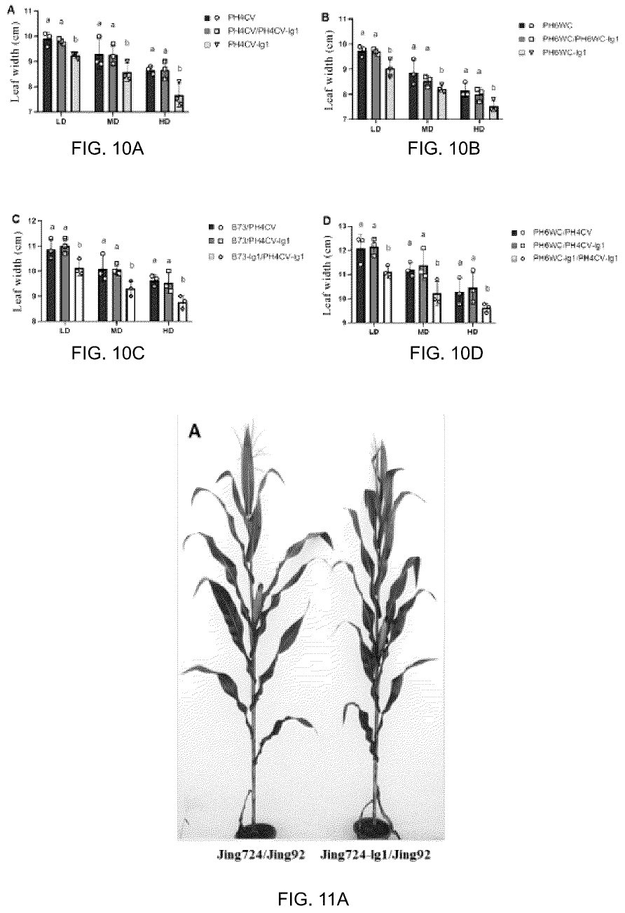

Then the LG1/LG1 wild-type material, the LG1/Ig1 heterozygous genotype material and the Ig1/Ig1 homozygous mutant material under the background of the PH6WC, PH4CV and B73 inbred lines were subjected to strict density experiments in the field. The experiment was carried out in Langfang Experimental Station, Hebei Province in 2022. Three planting densities were set for each material, which were 3000 plants/mu, 6000 plants/mu and 9000 plants/mu, respectively. Each material and density were planted in three replicates and four rows, and the middle two rows were selected to examine various agronomic characters. The phenotype analysis shows that the LG1/Ig1 heterozygous genotype material has a leaf angle and a tassel branch number which are both between those of the Ig1/Ig1 homozygous mutant material and the LG1/LG1 wild-type material under backgrounds of different inbred lines and different planting density conditions (FIGS. 4 and 5, statistics of the materials under the background of B73 were not obtained repeatedly); while having a normal auricle and ligule, thus can effectively prevent pests and pathogens from invading sheaths. In addition, we find that under backgrounds of different inbred lines and different planting density conditions, the Ig1/Ig1 homozygous mutant material has an empty stalk ratio which is significantly higher than both the LG1/Ig1 heterozygous genotype material and the LG1/LG1 wild-type material, and has a leaf width which is significantly narrower than both the LG1/Ig1 heterozygous genotype material and the LG1/LG1 wild-type material, indicating that the LG1 gene also plays an important role in regulating the empty stalk ratio and leaf width of maize. More interestingly, we find that under different inbred line materials, under the same planting density condition, there are no significant differences in the empty stem ratio and leaf width between those of the LG1/Ig1 heterozygous genotype material and the LG1/LG1 wild-type material (FIG. 8A, 8B, 8C, FIGS. 10A and 10B). The LG1/Ig1 heterozygous genotype material has a compacter leaf angle, and a reduced tassel branch number, and does not have an increased empty stalk ratio and a narrower leaf width, indicating that the LG1/Ig1 heterozygous genotype material has an unexpected use potential for density-tolerant breeding.

### Example 4. Test of Density-Tolerant Effect of LG1/LG1 Wild-Type Material, LG1/Ig1 Heterozygous Genotype Material and Ig1/Ig1 Homozygous Mutant Material Under Background of a Plurality of Hybrids

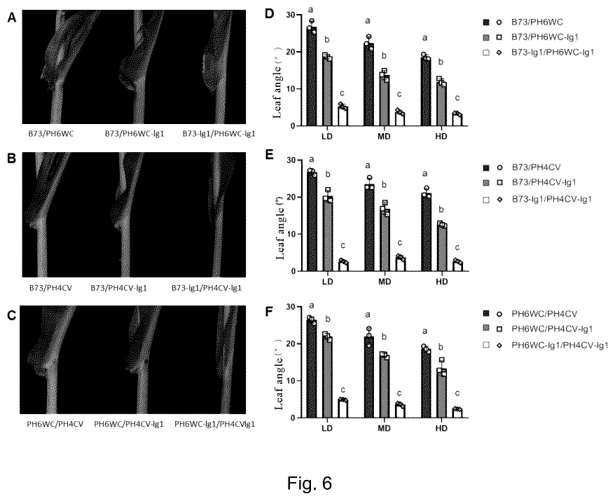

In order to test the use potential and universality of an LG1/Ig1 heterozygous genotype material in density-tolerant breeding of maize hybrids, different LG1 genetic materials under the background of the PH6WC, PH4CV and B73 inbred lines were hybridized to create different hybrid combinations. Then the LG1/LG1 wild-type material, the LG1/Ig1 heterozygous genotype material and the Ig1/Ig1 homozygous mutant material under the background of the hybrids B73/PH6WC, B73/PH4CV and PH6WC/PH4CV were subjected to strict density experiments in the field. The experiment was carried out in Langfang Experimental Station, Hebei Province in 2022. Three planting densities were set for each material, which were 3000 plants/mu, 5000 plants/mu and 7000 plants/mu, respectively. Each material and density were planted in three replicates and four rows, and the middle two rows were selected to examine various agronomic characters. The phenotype analysis shows that similar to inbred lines, under backgrounds of different hybrids and different planting density conditions, the LG1/Ig1 heterozygous genotype material has a leaf angle, a tassel branch number and a tassel branch angle which are all between those of the Ig1/Ig1 homozygous mutant material and the LG1/LG1 wild-type material (FIGS. 6 and 7, statistics of tassel branch number of the material under the background of the PH6WC/PH4WC hybrid were not obtained), while having a normal auricle and ligule, thus can effectively prevent pests and pathogens from invading sheaths; the Ig1/Ig1 homozygous mutant material has an empty stalk ratio which is significantly higher than both that of the LG1/Ig1 heterozygous genotype material and that of the LG1/LG1 wild-type material, and has a leaf width which is significantly narrower than both that of the LG1/Ig1 heterozygous genotype material and that of the LG1/LG1 wild-type material under backgrounds of different inbred lines and different planting density conditions, and there are no significant differences in the empty stem ratio and leaf width between those of the LG1/Ig1 heterozygous genotype material and the LG1/LG1 wild-type material (FIG. 8D, 8E, 8F, FIGS. 10C and 10D).

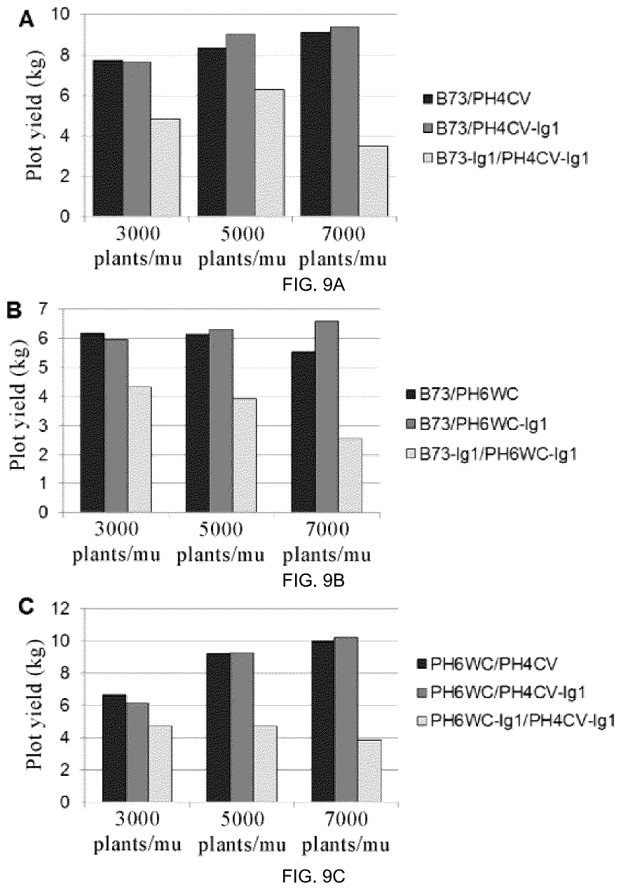

The plot yields (yield per unit area) of different hybrid materials were investigated, and it is found that under different hybrids and different density conditions, the Ig1/Ig1 homozygous mutant has a plot yield which is significantly lower than that of the LG1/LG1 wild-type material, and even lower under high density conditions (FIG. 9), indicating that the Ig1/Ig1 homozygous mutant cannot be really used for conventional yield breeding and density-tolerant breeding of maize, despite its superior plant type. Under low density conditions, the LG1/Ig1 heterozygous genotype material has a plot yield which is slightly lower than that of the LG1/LG1 wild-type material, but with the increase of planting density, will gradually exceed that of the LG1/LG1 wild-type material; under high density conditions (≥5000 plants/mu or above), the LG1/Ig1 heterozygous genotype material has a plot yield which increases significantly compared with that of the LG1/LG1 wild-type material (FIG. 9), indicating that the material has the effect of increasing the yield by close planting and is suitable for different hybrid backgrounds.

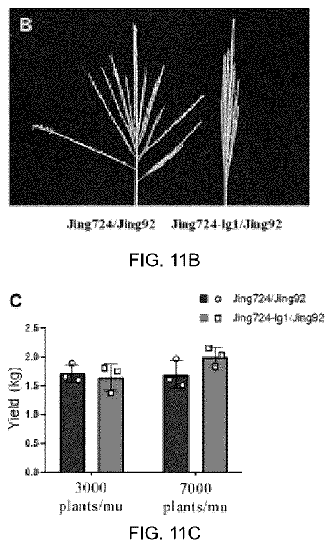

In addition, we also used Jing724, Jing724-Ig1 and Jing92 for hybridization to create the hybrids Jing724/Jing92 and Jing724-Ig1/Jing92. A similar density experiment was carried out in Langfang Experimental Station, Hebei Province in 2023. Two planting densities were set for each material, which were 3000 plants/mu, and 7000 plants/mu, respectively. Each material and density were planted in three replicates and four rows, and the middle two rows were selected to examine various agronomic characters. Phenotype and yield analyses show that the LG1/Ig1 heterozygous genotype material has a leaf angle, a tassel branch number and a tassel branch angle which are significantly smaller than those of the LG1/LG1 wild-type material (FIG. 11), and the LG1/Ig1 heterozygous genotype material has a normal auricle and ligule, thus can effectively prevent pests and pathogens from invading sheaths; under low density conditions, the LG1/Ig1 heterozygous genotype material has a plot yield which is comparable to that of the LG1/LG1 wild-type material, but under high density conditions (≥7000 plants/mu or above), the LG1/Ig1 heterozygous genotype material has a plot yield which increases significantly compared with that of the LG1/LG1 wild-type material.

In conclusion, we have proved that the LG1/Ig1 heterozygous genotype material (compared with the LG1/LG1 wild-type material) has effects of making a leaf angle compacter, reducing a tassel branch number and making a tassel branch angle smaller, while keeping a leaf width from narrowing and keeping an empty stalk ratio from increasing in maize under different density conditions, and the LG1/Ig1 heterozygous genotype material can significantly increase the yield per unit area of maize hybrids under high density conditions (≥5000 plants/mu).

What is claimed is:

1. A method for improving density-tolerant breeding of maize, comprising: hybridizing an Ig1 mutant strain with an LG1 wild-type strain to obtain an LG1/Ig1 heterozygous genotype strain, wherein compared with the LG1 wild-type strain, the heterozygous genotype strain has excellent density-tolerant characters of a reduced leaf angle, a reduced tassel branch number, a reduced tassel angle and/or an increased yield by close planting; or, compared with the Ig1 mutant strain, the heterozygous genotype strain has excellent density-tolerant characters of a widened leaf, a larger spike, an increased tassel angle, a reduced empty stalk ratio and/or an increased yield by close planting;
the polynucleotide sequence of the LG1 wild-type gene is selected from one of the sequences in following groups:
(a) a polynucleotide sequence as shown in SEQ ID No: 1, 2 or 3;
(b) a polynucleotide sequence encoding an amino acid sequence as shown in SEQ ID No: 4;
(c) a polynucleotide sequence which can hybridize with the polynucleotide sequence in (a) or (b) under stringent hybridization conditions, wherein the homozygous mutation of the polynucleotide sequence has functions of completely deleting ligule and auricles, reducing a leaf angle, making a leaf erect, narrowing a leaf, reducing a tassel branch number, reducing a tassel branch angle, raising an empty stalk ratio and/or making a spike smaller in a plant;
(d) a polynucleotide sequence which has at least 90%, 95%, 98% or more sequence identity to the polynucleotide sequence as shown in any one of (a) to (c), wherein the homozygous mutation of the polynucleotide sequence has functions of completely deleting a ligule and an auricle, reducing a leaf angle, making a leaf erect, narrowing a leaf, reducing a tassel branch number, reducing a tassel branch angle, raising an empty stalk ratio and/or making a spike smaller in a plant; or
(e) a polynucleotide sequence which is fully complementary to the sequence of any one of (a) to (d).

2. The method of claim 1, wherein the Ig1 mutant strain is obtained by mutation comprising substitution, deletion and/or addition of one or more nucleotides on the nucleotide sequence of the gene.

3. The method of claim 2, wherein the mutation is obtained by natural mutation, physical mutagenesis, chemical mutagenesis, ZFN, TALEN and/or CRISPR/Cas gene editing technology.

4. The method of claim 1, wherein the Ig1 mutant has a genomic mutant nucleotide sequence selected from one of the sequences in following groups:
(a) a genomic mutant nucleotide sequence in which the whole nucleotide sequence of SEQ ID No.1 is deleted;
(b) a genomic mutant nucleotide sequence in which the nucleotide sequence at 743-764 bp of SEQ ID No.1 or SEQ ID No.2 is replaced by
5′-TTCCGCCGCCGTCC-3′;
(c) a genomic mutant nucleotide sequence in which a single nucleotide deletion occurs at 836 bp of SEQ ID No.1 or SEQ ID No.2; or
(d) a genomic mutant nucleotide sequence in which a sequence deletion occurs at 766-835 bp of SEQ ID No.1 or SEQ ID No.2; or
(e) a genomic mutant nucleotide sequence in which the nucleotide sequence at 822-837 bp of SEQ ID No.1 or SEQ ID No.2 is replaced by 5′-GGA-3′.

5. The method of claim 1, wherein the close planting means more than or equal to 4000 plants/mu.

6. Use of the method of any one of claim 1 in reducing a leaf angle, widening a leaf, reducing a tassel branch number, reducing a tassel branch angle, reducing an empty stalk ratio and/or increasing a yield by close planting in a plant.

7. The use of claim 6, wherein the increased yield by close planting means increasing a maize yield under a high density condition, and the high density means more than or equal to 4000 plants/mu.

8. A method for regulating and controlling agronomic characters of a maize plant by obtaining a homozygous mutant through mutating an endogenous gene of the plant, wherein the homozygous mutant has phenotypes of a completely deleted ligule and auricle, a reduced leaf angle, an erect leaf, a narrowed leaf, a reduced tassel branch number, a reduced tassel branch angle, a raised empty stalk ratio and/or a smaller spike, and the polynucleotide sequence of the endogenous gene is selected from one of the sequences in following groups:
(a) a polynucleotide sequence as shown in SEQ ID No: 1, 2 or 3;
(b) a polynucleotide sequence encoding an amino acid sequence as shown in SEQ ID No: 4;
(c) a polynucleotide sequence which can hybridize with the polynucleotide sequence in (a) or (b) under stringent hybridization conditions, wherein the homozygous mutation of the polynucleotide sequence has functions of completely deleting a ligule and an auricle, reducing a leaf angle, making a leaf erect, narrowing a leaf, reducing a tassel branch number, reducing a tassel branch angle, raising an empty stalk ratio and/or making a spike smaller in a plant;
(d) a polynucleotide sequence which has at least 90%, 95%, 98% or more sequence identity to the polynucleotide sequence as shown in any one of (a) to (c), wherein the homozygous mutation of the polynucleotide sequence has functions of completely deleting a ligule and an auricle, reducing a leaf angle, making a leaf erect, narrowing a leaf, reducing a tassel branch number, reducing a tassel branch angle, raising an empty stalk ratio and/or making a spike smaller in a plant; or
(e) a polynucleotide sequence which is fully complementary to the sequence of any one of (a) to (d).


SEQUENCE LISTING (7 sequences)

SEQ ID NO: 1 - DNA, 4011 bp, Zea mays
  source 1..4011: mol_type = genomic DNA
actcactccaccaccactcgttccatggaaccctagaccaaaagtactcgcgctctcggctctcgctctcgccctctccctctccgcagcaaaagagatccggccggccgagaagggcgcgcgctagctgcccggctactagctggcgcccgcccgcgcatatatctgtgtcatcgccatcacccacaccatggcccggccggccaacaccgccgtattagctctgtctgtcgctcgtccacctgcgaccgactgagcgatcgatctccaccgagctctccgctaagcgctgtccttgccgccgtcctcccctccgtcccctacgcatccatttccgtgtgctcgtgtgtgcgcgcgcgggcactcctgctcctgctccctccggcccctcctcccctcccaggctcccagctagccgcgcccgcccgcgcgacctgcacctgcacagatcgggcggccgggccgaccgatcgatcgagatatcgtgtcaacgtgccggccggggcgtgggaagatgatgaacctatcggctgccgccaacggccgcgacgagttccccccctacgtcgtgccgtccaacgcggccgctccgcccccttccctgctcccaaccatggagcagcagcaggagagcagcatccacagggagcatcatcagctgctgggctacaacctcgaggccaactcgctggccctcctgcccccgtccaacgccgccgccgcccaccaccacaccaccttcgccggcggccacagcccccacgacatcctccacttctaccctcctccttccgccgcctcgcactacctcgccgccgccggcggcaacccctacagccacttagtctccgcgcccgggaccaccttccaccagacctcgtcgtcctactacccgccggcggcggcggcgcaggccgcgcccgagtactacttccccaccctcgtcagctccgccgaggagaacatggccagcttcgccgccacgcagctcggcctcaacctcggctaccgcacctacttcccgcccagaggaggctacacgtacggccaccacccgccgcgctgccaggccgagggctgcaaggccgacctctccagcgccaagagataccaccgtcgccacaaggtgtgcgagcaccactccaaggcgcccgtcgtcgtcaccgccggtggactgcatcagaggttctgccagcagtgcagcaggtcagtaggccactcggtcatcactggatccaattcaattcaattcaattcaatccatcgatctcgatctctctggctagccaggttctcaacttaattcgaggggtaaataatctacctttatggttcggtcgggtacttcacatgtctatatagatcttgtctacacatacatatatatctgaaatcatcacgagttgcattaagtgatccccccttcagttcattggccagcttgatcgaaactgatttcatgcatgctttattgttgctcgatctcacgagttatctggcactcttcgaagctacaattttgtcagagaaatcttttgggaggataggttacttcgttgcgcctcatgatacatcacttatagtttgcatgatatcataattatttccttcttttttagttataaaaagtcaattgaacaagggggtgaaagctattgcttttattttgcatgatatcatccctgcccttatttctctctgacttcgtctcttcagctgcattttaaccgaatatatctcagtttgacatgttatgcgtttcgatatatatgactccatttcatgagcctaatttccccagaaaccttatatttcttgaggcaaactttctaaaatgactgtactcctacttatattagcaaccactacttctcgatctatatgtattatattattatataacaattgaatccaacaacaaactaaagagagcatgcttaattgacaaaaatccagttgttattaacacaaaatgcataaatatgtacttactggttgcaaagcaaaagatctgtaagccattttccctcgtcagttcacatcccagttaataatttgccgctgaaattatctagccctttctcttcaacattttggtaaacctagattttcttttgtctctcctcctcctcctcttccactcaatttcttccttggcattatttactcctttccattttatttcttccttggcattactcgtttccatttatttccttgacttctccgttcttggtattttcgatctagtagttattataaacagcgaggtggctgtacaagtattggcctctcaaaaaagtcagaaacaaggctactttcttccaagcatcgcaagactttgtacagccgccgggtagacgtacgtacgtacgtacgagcaaaaattgcaaatgcaagcaacaatgccttgttagcttctctctactctctctctctctctctctacctctctctctctctcatgtattatttctctttcgtatcaattggctggtatagtagctagcaatccgttggccagagaagagacatacatacgttctgttaccggccaatataatccatgggctggccggacctgcaaatagatctcgtctgaggagcatgcacgtgtgaagtaataaccggcaatgcatttaatggtaaacaggatctctcccagaatgattcaccacattgagacacgccatatgtgctcagctgcaaaactccaagctgtctgcagggaagaacagtggagtatagatacatgttcctcatgttctggatcatcatcattagtaataacgcgcatgccattggtccccggaatgttccgtttttacttgagggaaatggactcagatctccctcttccatcacgcatgtacgaggcatgcatcatcagctcatcttgcattaatattgctatataatccaactacagtgtataattatatgaatgatatatactgcatgcatggctttcctctggatacgtggtatttcatgtgtatggatgctttcgtaatacccttgcttcaattctttttctctttttcctcgcacacctttgtctctcagattccatctgctggatgagttcgacgatgctaagaagagctgcaggaaacgcctagcggaccacaaccgccgccgccggaagtcaaagccatcggatgctgatgccggagacaagaaaagggcacatgcgaacaaagcagctgctgctaaagacagtacgtgatctttctacactcttttgttattactgttgtatgtcttcaaaaaaactttcctctttttattgagagactgaacacacaaggtgcttagttgtgcactacacactacgacatccacatgcatgcatataatcgacctatatatatatagcatgttcatcttagtagtaacatcgacaccgtttccttggaatgaaaatctcaacagaagcagagagtagcagcaagaacatggatatcggagatgggttgggcgcacagatactgggaagtgcactcttgtccaaggaacaagatcaagccatggatcttggagaggtggtgaaagaagcagtggatcccaaggggaaggcatcaatgcaacagcattacggcttccccttccattcgtcgtcagcaggatcttgcttcccccagacccaagccgtctccagtgataccacatccaatataggtcaagtgcaagagccgagcttagggttccaccatcatcagcaccaccaacacagcaacatcttgcagctcggccaggctatgtttgatctcgacttcgatcactagtcaatatgtgatgcacatgcaccctctctttctcaccccacccctccctccctctatcttgtttgtgcgcgtaatccgaatatttttcctttttaaattatctgtgttaattactgtaacgtggacataataatgatagtctatgcttgccatgcaatcatctatattatagttaatcattgtttcattgctcattatagtagtacttcttgcttcacatgtg

SEQ ID NO: 2 - DNA, 1937 bp, Zea mays
  source 1..1937: mol_type = other DNA
actcactccaccaccactcgttccatggaaccctagaccaaaagtactcgcgctctcggctctcgctctcgccctctccctctccgcagcaaaagagatccggccggccgagaagggcgcgcgctagctgcccggctactagctggcgcccgcccgcgcatatatctgtgtcatcgccatcacccacaccatggcccggccggccaacaccgccgtattagctctgtctgtcgctcgtccacctgcgaccgactgagcgatcgatctccaccgagctctccgctaagcgctgtccttgccgccgtcctcccctccgtcccctacgcatccatttccgtgtgctcgtgtgtgcgcgcgcgggcactcctgctcctgctccctccggcccctcctcccctcccaggctcccagctagccgcgcccgcccgcgcgacctgcacctgcacagatcgggcggccgggccgaccgatcgatcgagatatcgtgtcaacgtgccggccggggcgtgggaagatgatgaacctatcggctgccgccaacggccgcgacgagttccccccctacgtcgtgccgtccaacgcggccgctccgcccccttccctgctcccaaccatggagcagcagcaggagagcagcatccacagggagcatcatcagctgctgggctacaacctcgaggccaactcgctggccctcctgcccccgtccaacgccgccgccgcccaccaccacaccaccttcgccggcggccacagcccccacgacatcctccacttctaccctcctccttccgccgcctcgcactacctcgccgccgccggcggcaacccctacagccacttagtctccgcgcccgggaccaccttccaccagacctcgtcgtcctactacccgccggcggcggcggcgcaggccgcgcccgagtactacttccccaccctcgtcagctccgccgaggagaacatggccagcttcgccgccacgcagctcggcctcaacctcggctaccgcacctacttcccgcccagaggaggctacacgtacggccaccacccgccgcgctgccaggccgagggctgcaaggccgacctctccagcgccaagagataccaccgtcgccacaaggtgtgcgagcaccactccaaggcgcccgtcgtcgtcaccgccggtggactgcatcagaggttctgccagcagtgcagcagattccatctgctggatgagttcgacgatgctaagaagagctgcaggaaacgcctagcggaccacaaccgccgccgccggaagtcaaagccatcggatgctgatgccggagacaagaaaagggcacatgcgaacaaagcagctgctgctaaagacaaagcagagagtagcagcaagaacatggatatcggagatgggttgggcgcacagatactgggaagtgcactcttgtccaaggaacaagatcaagccatggatcttggagaggtggtgaaagaagcagtggatcccaaggggaaggcatcaatgcaacagcattacggcttccccttccattcgtcgtcagcaggatcttgcttcccccagacccaagccgtctccagtgataccacatccaatataggtcaagtgcaagagccgagcttagggttccaccatcatcagcaccaccaacacagcaacatcttgcagctcggccaggctatgtttgatctcgacttcgatcactagtcaatatgtgatgcacatgcaccctctctttctcaccccacccctccctccctctatcttgtttgtgcgcgtaatccgaatatttttcctttttaaattatctgtgttaattactgtaacgtggacataataatgatagtctatgcttgccatgcaatcatctatattatagttaatcattgtttcattgctcattatagtagtacttcttgcttcacatgtg

SEQ ID NO: 3 - DNA, 1200 bp, Zea mays
  source 1..1200: mol_type = other DNA
atgatgaacctatcggctgccgccaacggccgcgacgagttccccccctacgtcgtgccgtccaacgcggccgctccgcccccttccctgctcccaaccatggagcagcagcaggagagcagcatccacagggagcatcatcagctgctgggctacaacctcgaggccaactcgctggccctcctgcccccgtccaacgccgccgccgcccaccaccacaccaccttcgccggcggccacagcccccacgacatcctccacttctaccctcctccttccgccgcctcgcactacctcgccgccgccggcggcaacccctacagccacttagtctccgcgcccgggaccaccttccaccagacctcgtcgtcctactacccgccggcggcggcggcgcaggccgcgcccgagtactacttccccaccctcgtcagctccgccgaggagaacatggccagcttcgccgccacgcagctcggcctcaacctcggctaccgcacctacttcccgcccagaggaggctacacgtacggccaccacccgccgcgctgccaggccgagggctgcaaggccgacctctccagcgccaagagataccaccgtcgccacaaggtgtgcgagcaccactccaaggcgcccgtcgtcgtcaccgccggtggactgcatcagaggttctgccagcagtgcagcagattccatctgctggatgagttcgacgatgctaagaagagctgcaggaaacgcctagcggaccacaaccgccgccgccggaagtcaaagccatcggatgctgatgccggagacaagaaaagggcacatgcgaacaaagcagctgctgctaaagacaaagcagagagtagcagcaagaacatggatatcggagatgggttgggcgcacagatactgggaagtgcactcttgtccaaggaacaagatcaagccatggatcttggagaggtggtgaaagaagcagtggatcccaaggggaaggcatcaatgcaacagcattacggcttccccttccattcgtcgtcagcaggatcttgcttcccccagacccaagccgtctccagtgataccacatccaatataggtcaagtgcaagagccgagcttagggttccaccatcatcagcaccaccaacacagcaacatcttgcagctcggccaggctatgtttgatctcgacttcgatcactag

SEQ ID NO: 4 - protein, 399 aa, Zea mays
MMNLSAAANGRDEFPPYVVPSNAAAPPPSLLPTMEQQQESSIHREHHQLLGYNLEANSLALLPPSNAAAAHHHTTFAGGHSPHDILHFYPPPSAASHYLAAAGGNPYSHLVSAPGTTFHQTSSSYYPPAAAAQAAPEYYFPTLVSSAEENMASFAATQLGLNLGYRTYFPPRGGYTYGHHPPRCQAEGCKADLSSAKRYHRRHKVCEHHSKAPVVVTAGGLHQRFCQQCSRFHLLDEFDDAKKSCRKRLADHNRRRRKSKPSDADAGDKKRAHANKAAAAKDKAESSSKNMDIGDGLGAQILGSALLSKEQDQAMDLGEVVKEAVDPKGKASMQQHYGFPFHSSSAGSCFPQTQAVSSDTTSNIGQVQEPSLGFHHHQHHQHSNILQLGQAMFDLDFDH

SEQ ID NO: 5 - DNA, 23 bp, synthetic construct
  source 1..23: mol_type = other DNA
gcggagactaagtggctgtaggg

SEQ ID NO: 6 - DNA, 23 bp, synthetic construct
  source 1..23: mol_type = other DNA
gggggagcatcaccatcaactgc

SEQ ID NO: 7 - DNA, 14 bp, unidentified
  source 1..14: mol_type = other DNA
ttccgccgccgtcc